(19) United States
(12) Patent Application Publication
(10) Pub. No.: US 2020/0230433 A1
(43) Pub. Date: Jul. 23, 2020

(54) PHOTOTHERAPY GARMENT FOR TREATING NEONATAL JAUNDICE AND OTHER MEDICAL CONDITIONS

(71) Applicant: DUPONT ELECTRONICS, INC., Wilmington, DE (US)

(72) Inventors: H DAVOD ROSENFELD, DRUMORE, PA (US); JEFFREY M CALVERT, ACTON, MA (US)

(73) Assignee: ROHM AND HAAS ELECTRONIC MATERIALS LLC, MARLBOROUGH, MA (US)

(21) Appl. No.: 16/750,116

(22) Filed: Jan. 23, 2020

Related U.S. Application Data
(60) Provisional application No. 62/795,879, filed on Jan. 23, 2019.

(51) Int. Cl. A61N 5/06; A41D 13/12

(52) U.S. Cl. CPC A61N 5/0621; A61N 2005/0645; A41D 13/1272

(57) ABSTRACT

This invention provides a phototherapy garment for treating neonatal jaundice, the phototherapy garment comprising a first layer of a flexible soft-textured non-woven material, a second layer of a flexible stretchable substrate supporting light-emitting diode (LED) light sources and a thin flexible third layer with heat transfer capability to provide dissipation of waste heat from the LEDs of the second layer.

This invention also provides a phototherapy garment for treating other medical conditions.

### CROSS-REFERENCE TO RELATED APPLICATION

This application claims benefit under 35 USC 119(e) of U.S. Provisional Application Ser. No. 62/795,879, filed Jan. 23, 2019, which application is incorporated herein for all purposes by reference thereto.

### FIELD OF THE INVENTION

This invention is directed to garments that expose jaundiced infants and individuals with other medical conditions to phototherapy.

### BACKGROUND OF THE INVENTION

Phototherapy has been used to treat neonatal jaundice. The treatment involves exposing the infant to very bright blue light for extended periods of time to break down the bilirubin. The infant is unclothed but blindfolded during the exposure to maximize the amount of light delivered to the skin while protecting the eyes. This is an uncomfortable situation for both the infant and the infant's parents. Alternate treatments have been proposed but there is need for a lower cost and convenient approach that can be used in the infant's home, wherein the phototherapy equipment is fully contained in a comfortable, washable garment.

Phototherapy has also been used to treat skin conditions such as acne, eczema and psoriasis and sleep and mood disorders. and convenient treatments are needed.

### SUMMARY OF THE INVENTION

This invention provides a phototherapy garment for a human subject, such as an infant, at least a portion of the garment being formed of a multi-layer cloth comprising:
a) a first layer of a flexible material;
b) a second layer comprising a flexible substrate and a light source and conductors supported thereon, the conductors being formed using a stretchable polymer thick film paste containing an electrically conductive material and connected to the light source; and
c) a flexible third layer with heat transfer capability to provide dissipation of waste heat from the light source of the second layer,
and wherein the first layer is adjacent to the subject's body and diffuses light from the light source onto the body.

This invention also provides a method for treating neonatal jaundice in an infant, comprising:
a) providing a phototherapy garment configured for the infant, at least a portion of the garment being formed of a multi-layer cloth comprising:
   (i) a first layer of a flexible material;
   (ii) a second layer comprising a flexible substrate comprising a light source and conductors supported thereon, the conductors being formed using a stretchable polymer thick film paste containing an electrically conductive material and connected to the light source, the light source operating between 420 nm and 520 nm with a spectral irradiance of between 25 and 50 μW/cm²/nm; and
   (iii) a flexible third layer with heat transfer capability to provide dissipation of waste heat from the light source of the second layer
b) situating the infant in the phototherapy garment; and
c) activating the light source for a duration that is therapeutically effective for treating the neonatal jaundice.

### BRIEF DESCRIPTION OF THE DRAWINGS

The invention will be more fully understood and further advantages will become apparent when reference is made to the following detailed description of the preferred embodiments of the invention and the accompanying drawings, wherein like reference numerals denote similar elements throughout the several views and in which:

FIG. 1 depicts in expanded, perspective view a multi-layer fabric useful in constructing a phototherapy garment of the present invention; and

FIG. 2 depicts a phototherapy garment of the present invention suitable for an infant.

### DETAILED DESCRIPTION OF THE INVENTION

This invention relates in an aspect to a phototherapy garment for an infant. The garment should be stretchable and flexible in order to be comfortable to the infant and to accommodate wearing, folding, handling, washing, and drying. The garment should fit closely to the body of the infant so that the light is essentially contained within the garment and the infant's eyes are not exposed to the light. The garment may be configured in any suitable form, including, without limitation, a hooded one-piece pajama that optionally covers the hands and feet, rompers, a sweater with or without a hood, a jacket with or without a hood, pants with or without covering for feet, a sac, or a blanket.

Some or all of the garment is constructed using a multi-layer cloth that comprises: a first layer of material that is preferably flexible and soft-textured; a second layer of a flexible substrate supporting a source of suitable light, such as a plurality of light-emitting diode (LED) light sources; and a third layer that preferably is reflective and has heat transfer capability to provide dissipation of waste heat from the light source(s) of the second layer. The garment is configured such that the first layer is adjacent the skin of a wearer. The layers may be permanently secured to each other by any suitable attachment including, without limitation, adhesive lamination, sewing, quilting, or the like. Alternatively, the layers may be detachably secured to each other by buttons, snaps, zippers, or other like fasteners.

In an embodiment, some of the garment is constructed with panels of the foregoing lighted multi-layer cloth. Since the torso includes a large fraction of the wearer's body surface, it is preferred that a substantial fraction of at least the torso portion include the lighted cloth, while other portions, such as the openings for the head, arms, and legs may be made of a secondary material, such as one that is elasticized to secure the garment and inhibit light leakage. Other embodiments provide a garment in which additional portions include suitable light sources.

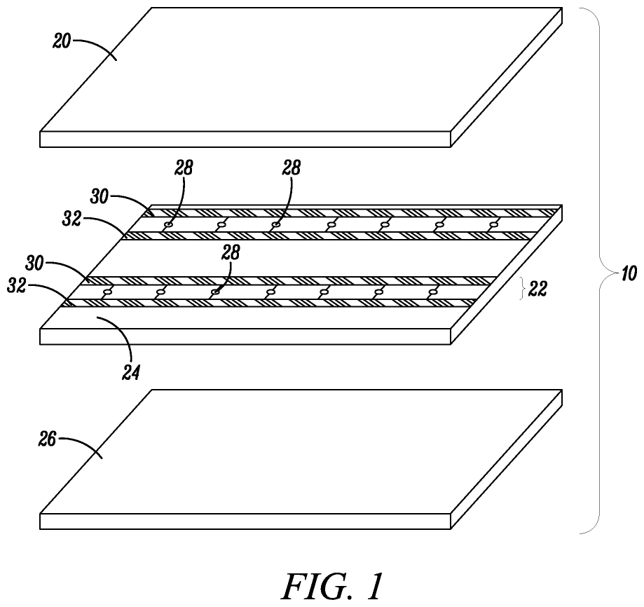

FIG. 1 depicts an expanded view of a form of multilayer cloth 10 suitably used in the construction of the present garment, including first layer 20, second layer 22, and third layer 26.

First layer 20 comprises a flexible material that may be either woven or non-woven. A suitable non-woven material may be a sheet of fibers, continuous filaments or chopped yarns that have been formed into a web and bonded together. Since the garment is ordinarily worn with first layer 20 adjacent to the infant's body, a soft texture is beneficial for the sake of comfort. Using a material for the inner layer that is pervious to moisture may further enhance comfort. First layer 20 is sufficiently transparent or translucent, so that it transmits or preferably diffuses most or all the light coming from the second layer 22 to impinge uniformly on the infant's skin.

Second layer 22 comprises a flexible, stretchable substrate 24 and conductors and a light source supported thereon. The light source may be of any type that provides sufficient output within the spectrum of interest without generating excessive heat. In some embodments an electroluminescent material is used. The embodiment shown in FIG. 1 comprises a plurality of LEDs 28 formed by a printing process. The light output spectrum of the LEDs or other light source and the spectral irradiance can be chosen depending upon the condition being treated. For treating infant jaundice, the LEDs beneficially emit light with wavelength between 420 nm and 520 nm with a spectral irradiance between 25 and 50 μW/cm²/nm or between 30 and 35 μW/cm²/nm. Alternatively, LEDs 28 may be conventional, rigid, discrete devices. LEDs 28 are energized through electrical connection to conductors 30, 32, which are printed on flexible substrate 24. For example, conductors 30, 32 may be formed from a stretchable polymer thick film paste containing an electrically conductive material. In one embodiment, the electrical conductors are formed using Intexar™ PE873 Stretchable Silver Conductor (DuPont Co., Wilmington, Del.) paste. Both LEDs 28 and conductors 30, 32 are printed on stretchable substrate 24. In one embodiment, the substrate 24 is thermoplastic polyurethane (TPU). In another embodiment, the substrate is polydimethylsiloxane (PDMS). Conductors 30, 32 are appointed to be connected to a source of electrical energy.

The third layer 26 is preferably thin and flexible. It is also preferably opaque and reflective, so that light generated by the source does not escape and preferably most or all of it is directed to reach the skin of the garment wearer. It is also preferred that the third layer 26 have heat transfer capability to provide dissipation of waste heat from the LEDs of the second layer. In one embodiment, the third layer is a metallized woven or non-woven fabric or polymer sheet.

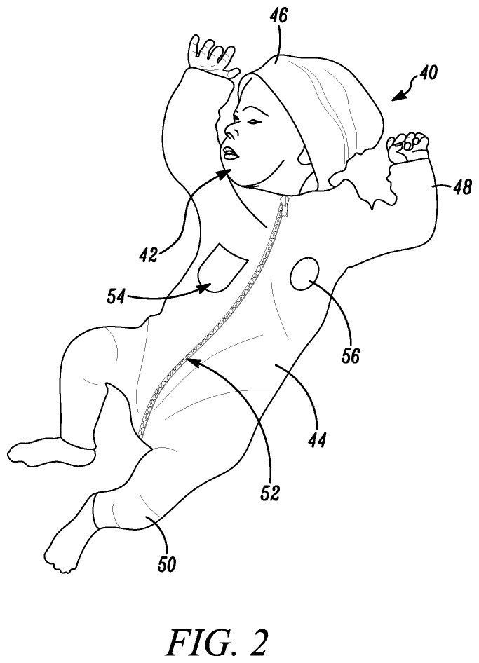

An exemplary embodiment of the present garment is depicted by FIG. 2, in which a phototherapy garment generally shown at 40 is worn by an infant 42. The implementation shown provides a pajama-like configuration that includes a torso portion 44, a hood 46, and arms 48 and legs 50. As shown, arms 48 are open, allowing the hands of the infant to exend freely, while each of legs 50 include an integral foot-enclosing portion to provide warmth and comfort. However, each of hood 46, arms 48, and legs 50 (and the associated hand- and foot-covering portions) is optional. For convenience, openable zipper 52 is provided to allow garment 40 to be placed comfortably and securely onto infant 42, so that treatment can be carried out without light emanating from any of the openings through which the head, neck, and extremities of the infant extend. Other forms of closure, such as buttons, hooks, ties, hook-and-loop fasteners (e.g., VELCRO™ strips) and adhesive strips, might also be used. Other garment configurations typical of those used for ordinary infant clothing of various types might also be used.

In one embodiment, the phototherapy garment employs a battery to supply power to the LEDs and associated circuitry. The garment may include a pocket, pouch, or the like, preferably with a closure, in which the battery is situated. FIG. 2 depicts a pouch 54 that may be configured to hold the battery, with suitable connections and wiring (not shown) being provided to connect the battery to conductors 30, 32. In another embodiment, the phototherapy garment comprises means to connect to an external power source to supply power to the LEDs.

In an embodiment, the phototherapy garment further comprises sensors for reflectance spectroscopy so that the skin color may be monitored. This information could be used to adjust the LEDs irradiance, i.e., the intensity and/or the spectral output, to optimize the phototherapy and make it a “smart” garment. In an embodiment, the garment may include circuitry that enables it to communicate with an external computer, smart phone, or the like device for any of monitoring the treatment subject and activating and controlling the light source. For example, the communication might be implemented using the Bluetooth protocol, as promulgated by the Bluetooth Special Interest Group, Kirkland, Wash.

Phototherapy has also been used to treat other medical conditions, including without limitation skin conditions (e.g., acne, eczema, and psoriasis) and sleep and mood disorders. In another aspect, this invention provides a phototherapy garment for an individual requiring such treatment. The garment in this aspect can be configured in a size and shape suitable for the patient being treated. The output spectrum and irradiance of the light source can likewise be provided in accordance with the condition to be treated.

Having thus described the invention in rather full detail, it will be understood that this detail need not be strictly adhered to but that further changes and modifications may suggest themselves to one skilled in the art, all falling within the scope of the invention as defined by the subjoined claims.

The embodiments of the phototherapy garment described herein, including the examples, are not limiting; it is contemplated that one of ordinary skill in the art could make minor substitutions and not substantially change the desired properties and its functioning in a system.

In this specification, unless explicitly stated otherwise or indicated to the contrary by the context of usage, where an embodiment of the subject matter hereof is stated or described as comprising, including, containing, having, being composed of, or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present. Additionally, the term “comprising” is intended to include examples encompassed by the terms “consisting essentially of” and “consisting of.” Similarly, the term “consisting essentially of” is intended to include examples encompassed by the term “consisting of.”

Certain terminology may be employed herein for clarity and convenience of description, rather than for any limiting purpose. For example, the terms “forward,” “rearward,” “right,” “left,” “top,” “bottom,” “upper,” and “lower” designate directions in the drawings to which reference is made. The various drawings may depict the present components oriented in a convenient configuration. Terminology of similar import other than the words specifically mentioned above likewise is to be considered as being used for purposes of convenience rather than in any limiting sense.

What is claimed is:

1. A phototherapy garment for a human subject, at least a portion of the garment being formed of a multi-layer cloth comprising:
a) a first layer of a flexible material;
b) a second layer comprising a flexible substrate and a light source and conductors supported thereon, the conductors being formed using a stretchable polymer thick film paste containing an electrically conductive material and connected to the light source; and
c) a flexible third layer with heat transfer capability to provide dissipation of waste heat from the light source of the second layer,
and wherein the first layer is adjacent to the subject's body and diffuses light from the light source onto the body.

2. The phototherapy garment of claim 1, wherein the garment is selected from the group consisting of a hooded one-piece pajama that covers hands and feet, rompers, a sweater with or without a hood, a jacket with or without a hood, pants with or without covering for feet, a sac, and a blanket.

3. The phototherapy garment of claim 1, wherein the third layer is a metallized fabric.

4. The phototherapy garment of claim 1, wherein the light source comprises a plurality of LEDs.

5. The phototherapy garment of claim 4, wherein the LEDs produce light having wavelengths between 420 nm and 520 nm with a spectral irradiance of between 25 and 50 μW/cm²/nm.

6. The phototherapy garment of claim 5, wherein the spectral irradiance is between 30 and 35 μW/cm²/nm.

7. The phototherapy garment of claim 4, wherein the LEDs are rigid, discrete devices.

8. The phototherapy garment of claim 4, wherein the LEDs are formed by a printing process.

9. The phototherapy garment of claim 1, further comprising a battery to supply power to the light source.

10. The phototherapy garment of claim 1, further comprising means to connect to an external power source to supply power to the light source.

11. The phototherapy garment of claim 1, further comprising sensors for reflectance spectroscopy.

12. The phototherapy garment of claim 1, configured for treating an infant.

13. A method for treating neonatal jaundice in an infant, comprising:
a) providing a phototherapy garment configured for the infant, at least a portion of the garment being formed of a multi-layer cloth comprising:
(i) a first layer of a flexible material;
(ii) a second layer comprising a flexible substrate comprising a light source and conductors supported thereon, the conductors being formed using a stretchable polymer thick film paste containing an electrically conductive material and connected to the light source, the light source operating between 420 nm and 520 nm with a spectral irradiance of between 25 and 50 μW/cm²/nm; and
(iii) a flexible third layer with heat transfer capability to provide dissipation of waste heat from the light source of the second layer
b) situating the infant in the phototherapy garment; and
c) activating the light source for a duration that is therapeutically effective for treating the neonatal jaundice.
